SEQUENCE LISTING (35 sequences)

SEQ ID NO: 1 - DNA, 1059 bp, Borrelia burgdorferi
  CDS (1)..(1059)
atgaaaattaaaagtaaatgcttagctttgggattgctttttggttttatcagttgtgatttattcataagagatgaaataaaagagaaatctcttggcttgtgtgatgaggaaagttctattttagagactggtgacaaatctgttaaaaagtctcttaataagaaaggcaaagataaggttgctagaaagaaagttgaaggtaatgctgttaaaaaagacccgtttaatcatcatgtaaagagggagtctgttaataatagtaatctatcacaaaaaaatgtgatatcggaagaagaaattttgaaaactaaattattaagagaacgacctgagactagaaaagaagaaatacaaaaacagcaagatgagcataaaaggatgcttcaaggaagtttaagttttcttagtggtgaaagtggtgaattgaaggatactattgaaagcaatgaaattgattttactatagattctgatttaagaccgaagagtgatttacaagctatttcaggctcaaattctatttcacatactgatgaaatagaagaagaagattatgatcagtattctttagaagaagattattattatgatgaggaaacaagattaagtaatagatatgaatcttatctagagggtgttaaatataatgtaagttcagcaattaaaacaattgttaagatatatgataattataccttactttcaacaaagcaaacccaaatgtattctacacgtcttgacaaccttgctaaagccaaagctagagaagaagctaaaaagtttacaaaagaagaacttgaaaaagatcttaagaccttattgaactatattcaagtgagtgcaaggactgcgacaaattttgtatatgcaagagaaatatattcaaaaagaaaattagatgccattgaaacagaaataaaaaatttaattttaaagatcaaaggacaatctgatttatacgaggcatataaagcaatagtaaggtcaatcttattaatgaaagattctcttaaaataatcgaaatagtcattgataagaatggtgtttggtactaa

SEQ ID NO: 2 - protein, 352 aa, Borrelia burgdorferi
MKIKSKCLALGLLFGFISCDLFIRDEIKEKSLGLCDEESSILETGDKSVKKSLNKKGKDKVARKKVEGNAVKKDPFNHHVKRESVNNSNLSQKNVISEEEILKTKLLRERPETRKEEIQKQQDEHKRMLQGSLSFLSGESGELKDTIESNEIDFTIDSDLRPKSDLQAISGSNSISHTDEIEEEDYDQYSLEEDYYYDEETRLSNRYESYLEGVKYNVSSAIKTIVKIYDNYTLLSTKQTQMYSTRLDNLAKAKAREEAKKFTKEELEKDLKTLLNYIQVSARTATNFVYAREIYSKRKLDAIETEIKNLILKIKGQSDLYEAYKAIVRSILLMKDSLKIIEIVIDKNGVWY

SEQ ID NO: 3 - DNA, 1059 bp, Borrelia burgdorferi
  CDS (1)..(1059)
atgaaaattaaaagtaaatgcttagctttgggattgctttttggttttatcagttgtgatttattcataagagatgaaataaaagagaaatctcttggcttgtgtgatgaggaaagttctattttagagactggtgacaaatctgttaaaaagtctcttaataagaaaggcaaagataaggttgctagaaagaaagttgaaggtaatgctgttaaaaaagacccgtttaatcatcatgtaaagagggagtctgttaataatagtaatctatcacaaaaaaatgtgatatcggaagaagaaattttgaaaactaaattattaagagaacgacctgagactagaaaagaagaaatacaaaaacagcaagatgagcataaaaggatgcttcaaggaagtttaagttttcttagtggtgaaagtggtgaattgaaggatactattgaaagcaatgaaattgattttactatagattctgatttaagactgaagagtgatttacaagctatttcaggctcaaattctatttcatatactgatgaaatagaagaagaagattatgatcagtattctttagaagaagattattattatgatggggaaacaagattaagtaatagatatgagtcttatctagagggtgttaaatataatgtaagttcagcaattaaaacaattgttaagatatatgataattataccttactttcaacaaagcaaacccaaatgtattctacacgtcttgacaaccttgctaaagccaaagctagagaagaagctaaaaagtttacaaaagaagaacttgaaaaagatcttaagaccttattgaactatattcaagtgagtgcaaggactgcgacaaattttgtatatgcaagagaaatatattcaaaaagaaaattagatgccattgaaacagaaataaaaaatttaattttaaagatcaaaggacaatctgatttatacgaggcatataaagcaatagtaaggtcaatcttattaatgaaagattctcttaaaataatcgaaatagtcattgataagaatggtgtttggtactaa

SEQ ID NO: 4 - protein, 352 aa, Borrelia burgdorferi
MKIKSKCLALGLLFGFISCDLFIRDEIKEKSLGLCDEESSILETGDKSVKKSLNKKGKDKVARKKVEGNAVKKDPFNHHVKRESVNNSNLSQKNVISEEEILKTKLLRERPETRKEEIQKQQDEHKRMLQGSLSFLSGESGELKDTIESNEIDFTIDSDLRLKSDLQAISGSNSISYTDEIEEEDYDQYSLEEDYYYDGETRLSNRYESYLEGVKYNVSSAIKTIVKIYDNYTLLSTKQTQMYSTRLDNLAKAKAREEAKKFTKEELEKDLKTLLNYIQVSARTATNFVYAREIYSKRKLDAIETEIKNLILKIKGQSDLYEAYKAIVRSILLMKDSLKIIEIVIDKNGVWY

SEQ ID NO: 5 - DNA, 1059 bp, Borrelia burgdorferi; identical to SEQ ID NO: 1
  CDS (1)..(1059)

SEQ ID NO: 6 - protein, 352 aa, Borrelia burgdorferi; identical to SEQ ID NO: 2

SEQ ID NO: 7 - DNA, 1059 bp, Borrelia burgdorferi
  CDS (1)..(1059)
atgaaaattaaaagtaaatgcttagctttgggattgctttttggttttatcagttgtgatttattcataagagatgaaataaaagagaaatctcttggcttgtgtgatgaggaaagttctattttagagactggtgacaaatctgttaaaaagtctcttaataagaaaggcaaagataaggttgctagaaagaaagttgaaggtaatgctgttaaaaaagacccgtttaatcatcatgtaaagagggagtctgttaataatagtaatctatcacaaaaaaatgtgatatcggaagaagaaattttgaaaactaaattattaagagaacgacctgagactagaaaagaagaaatacaaaaacagcaagatgagcataaaaggatgcttcaaggaagtttaagttttcttagtggtgaaagtggtgaattgaaggatactatagaaagcaatgaaattgattttactatagattctgatttaagactgaagagtgatttacaagctatttcaggctcaaattctatttcatatactgatgaaatagaagaagaagattatgatcagtattctttagaagaagattattattatgatgaggaaacaagattaagtaatagatatgaatcttatctagagggtgttaaatataatgtaagttcagcaattaaaacaattgttaagatatatgataattataccttactttcaacaaagcaaacccaaatgtattctacacgtcttgacaaccttgctaaagccaaagctagagaagaagctaaaaagtttacaaaagaagaacttgaaaaagatcttaagaccttattgaactatattcaagtgagtgcaaggactgcgacaaattttgtatatgcaagagaaatatattcaaaaagaaaattagatgccattgaaacagaaataaaaaatttaattttaaagatcaaaggacaatctgatttatacgaggcatataaagcaatagtaaggtcaatcttattaatgaaagattctcttaaaataatcgaaatagtcattgataagaatggtgtttggtactaa

SEQ ID NO: 8 - protein, 352 aa, Borrelia burgdorferi
MKIKSKCLALGLLFGFISCDLFIRDEIKEKSLGLCDEESSILETGDKSVKKSLNKKGKDKVARKKVEGNAVKKDPFNHHVKRESVNNSNLSQKNVISEEEILKTKLLRERPETRKEEIQKQQDEHKRMLQGSLSFLSGESGELKDTIESNEIDFTIDSDLRLKSDLQAISGSNSISYTDEIEEEDYDQYSLEEDYYYDEETRLSNRYESYLEGVKYNVSSAIKTIVKIYDNYTLLSTKQTQMYSTRLDNLAKAKAREEAKKFTKEELEKDLKTLLNYIQVSARTATNFVYAREIYSKRKLDAIETEIKNLILKIKGQSDLYEAYKAIVRSILLMKDSLKIIEIVIDKNGVWY

SEQ ID NO: 9 - DNA, 1083 bp, Borrelia burgdorferi
  CDS (1)..(1083)
atgaagaaagttaaaagtaaatatttagctttgggattactttttggttttataagttgtgatttattcataagatatgaaatgaaagaggaatcccctggcttatttgataagggaaactctattttagagactagcgaggaatctattaaaaagcctatgaataagaaaggcaaaggtaagattgccagaaagaacggaaaaagcaaggtttctggaaaagaaccgtttattcatagttttaaaagagacgctgctaataaaagcaattttttacaaaaaaatgtaatgttagaggaagaaagtttaaaaactgaattattaaaagagcaatctgagactagaaaagaaaaaatacaaaaacaacaagatgaatataaagggatgactaaaggaagtttaaattcccttagcggtgaaagtggtgaattgaaggagactattgaaagcaatgaaattgatattactatagattctgatttaaggccaaagagttccttacaagacattgcaggatcaaactctatttcatacactgatgaaatagaggaagaggattatgctcggtattatttagatgaagatgatgaagatgatgaatattatgaagatgattatgaggaaataagattaagcaatcgatatcaatcttatctagaaggtgttaaatataatgtagattcagcaattaacacaattaataagatatatgatacttatacattattctcaacaaagctaacccaaatgtattctacacgccttgacaaccttgctaaagccaaagctaaagaagaagctgcaaagtttacaaaagaagaccttgaaaaaaatttcaagaccttattgaattacattcaagtaagtgtaaagactgcaacaaattttgtatacataaatgaaatgcatgcaaaaaggaaattagagaacattgaagcaaaaataaaaactttaattgcaaagatcaaagaaaaatctaatttatactcagcatataaagcaatagtaagttcaatcttattaatgagggattctcttaaagaagtgcaatatgccattgacaagaatggcatttggtactaa

SEQ ID NO: 10 - protein, 360 aa, Borrelia burgdorferi
MKKVKSKYLALGLLFGFISCDLFIRYEMKEESPGLFDKGNSILETSEESIKKPMNKKGKGKIARKNGKSKVSGKEPFIHSFKRDAANKSNFLQKNVMLEEESLKTELLKEQSETRKEKIQKQQDEYKGMTKGSLNSLSGESGELKETIESNEIDITIDSDLRPKSSLQDIAGSNSISYTDEIEEEDYARYYLDEDDEDDEYYEDDYEEIRLSNRYQSYLEGVKYNVDSAINTINKIYDTYTLFSTKLTQMYSTRLDNLAKAKAKEEAAKFTKEDLEKNFKTLLNYIQVSVKTATNFVYINEMHAKRKLENIEAKIKTLIAKIKEKSNLYSAYKAIVSSILLMRDSLKEVQYAIDKNGIWY

SEQ ID NO: 11 - DNA, 1083 bp, Borrelia burgdorferi
  CDS (1)..(1083)
atgaagaaagttaaaagtaaatatttagctttgggattactttttggttttataagttgtgatttattcataagatatgaaatgaaagaggaatcccctggcttatttgataaggaaaactctattttagagactggcgaggaatctattaaaaagcctatgaataagaaagataaaggtaagattgccagaaagaaaggcaaaagcaaggtttctggaaaagaaccatctattcatagtttaaaaagggactctgctaataaaagcaattttttacaaaaaaatgtaatgttagaggaagaaagtttaaaaactgaattattaaaagagcaatctgagactagaaaagaaaaaatacaaaaacaacaagatgaatataaagggatgactaaaggaagtttaaattcccttagcggtgaaagtggtgaattgaaggagactattgaaagtaatgaaattgatattactatagattctgatttaaggctaaaaagtgccttacaaggcattgcaggatcaaactctatttcatacactgatgaaatagaggaagaggattatgatcggtattatttagaagaatatgatgaagatgttgaatattatgaagatgattatgaggaaataagattaagcaatcgatatcaatcttatctagaaggtgttaaatataatgtagattcagcaattaaaacaattaataagatatatgatacttatacattattttcaacaaagctaacccaaatgtattctacacgccttgacaaccttgctaaagccaaagctaaagaagaatctgcaaagtttacaaaagaagaccttgaaaaaaatttcaagaccttattgaattacattcaagtaagtgtaaagactgcaacaaattttgtatacataaatgacacgaccgcaaaaaggaaattagagaacattgaaacaaaaataaaaactttaattgcaaagatcaaagaaaaatctaatttatactcagcatataaagcaatagtaagttcaatcttattaatgagggattctcttaaagaagtgcaaagtgccattgacgagaatggcatttggtactaa

SEQ ID NO: 12 - protein, 360 aa, Borrelia burgdorferi
MKKVKSKYLALGLLFGFISCDLFIRYEMKEESPGLFDKENSILETGEESIKKPMNKKDKGKIARKKGKSKVSGKEPSIHSLKRDSANKSNFLQKNVMLEEESLKTELLKEQSETRKEKIQKQQDEYKGMTKGSLNSLSGESGELKETIESNEIDITIDSDLRLKSALQGIAGSNSISYTDEIEEEDYDRYYLEEYDEDVEYYEDDYEEIRLSNRYQSYLEGVKYNVDSAIKTINKIYDTYTLFSTKLTQMYSTRLDNLAKAKAKEESAKFTKEDLEKNFKTLLNYIQVSVKTATNFVYINDTTAKRKLENIETKIKTLIAKIKEKSNLYSAYKAIVSSILLMRDSLKEVQSAIDENGIWY

SEQ ID NO: 13 - DNA, 1083 bp, Borrelia burgdorferi; identical to SEQ ID NO: 9
  CDS (1)..(1083)

SEQ ID NO: 14 - protein, 360 aa, Borrelia burgdorferi; identical to SEQ ID NO: 10

SEQ ID NO: 15 - DNA, 1083 bp, Borrelia burgdorferi
  CDS (1)..(1083)
atgaaaaaagttaaaagtaaatatttggctttgggattactttttggttttataagttgtgatttattcataagatatgaaatgaaagaggaatcccctggcttatttgataagggaaactctattttagagactagcgaggaatctattaaaaagcctatgaataagaaaggtaaaggtaagattgctagaaagaaaggcaaaagcaaggtttctagaaaagaaccgtatattcatagtttaaaaagggactctgctaataaaagcaattttttacaaaaaaatgtaattttagaggaagaaagtttaaaaactgaattattaaaagagcaatctgagactagaaaagaaaaaatacaaaaacaacaagatgaatataaagggatgactcaaggaagtttaaattcccttagcggtgaaagtggtgaattgaaggagactattgaaagcaatgaaattgatattactatagattctgatttaaggccaaagagttccttacaagacattgcaggatcaaactctatttcatacactgatgaaatagaggaagaggattatgctcggtattatttagatgaagatgatgaagatgatgaatattatgaagatgattatgaggaaataagattaagcaatcgatatcaatcttatctagaaggtgttaaatataatgtagattcagcaattaacacaattaataagatatatgatacttatacattattctcaacaaagctaacccaaatgtattctacacgccttgacaaccttgctaaagccaaagctaaagaagaagctgcaaagtttacaaaagaagaccttgaaaaaaatttcaagaccttattaaattatattcaagtaagtgtaaagactgcagcaaattttgtatacataaatgacacgcatgcaaaaaggaaattagagaacattgaagcagaaataaaaactttaattgcaaagatcaaagaacaatctaatttatacgaagcatataaagcaatagtaacgtcaatcttattaatgagggattctcttaaagaagtgcaaggtatcattgacaagaatggcgtttggtactaa

SEQ ID NO: 16 - protein, 360 aa, Borrelia burgdorferi
MKKVKSKYLALGLLFGFISCDLFIRYEMKEESPGLFDKGNSILETSEESIKKPMNKKGKGKIARKKGKSKVSRKEPYIHSLKRDSANKSNFLQKNVILEEESLKTELLKEQSETRKEKIQKQQDEYKGMTQGSLNSLSGESGELKETIESNEIDITIDSDLRPKSSLQDIAGSNSISYTDEIEEEDYARYYLDEDDEDDEYYEDDYEEIRLSNRYQSYLEGVKYNVDSAINTINKIYDTYTLFSTKLTQMYSTRLDNLAKAKAKEEAAKFTKEDLEKNFKTLLNYIQVSVKTAANFVYINDTHAKRKLENIEAEIKTLIAKIKEQSNLYEAYKAIVTSILLMRDSLKEVQGIIDKNGVWY

SEQ ID NO: 17 - DNA, 20 bp, Borrelia burgdorferi
caccctcttgatagcactta

SEQ ID NO: 18 - DNA, 21 bp, Borrelia burgdorferi
ctttaaaggagagaaagcatg

SEQ ID NO: 19 - DNA, 31 bp, Borrelia burgdorferi
ccggatccgatttattcataagatatgaaat

SEQ ID NO: 20 - DNA, 20 bp, Borrelia burgdorferi
gcaatctgagactagaaaag

SEQ ID NO: 21 - DNA, 20 bp, Borrelia burgdorferi
tgcagtctttacacttactt

SEQ ID NO: 22 - DNA, 27 bp, Borrelia burgdorferi
ccctcgagattagtaccaaacgccatt

SEQ ID NO: 23 - DNA, 22 bp, Borrelia burgdorferi
acatattatgtagcctgtttta

SEQ ID NO: 24 - DNA, 21 bp, Borrelia garinii
ctttaaaggagagaaagcatg

SEQ ID NO: 25 - DNA, 31 bp, Borrelia garinii; identical to SEQ ID NO: 19

SEQ ID NO: 26 - DNA, 20 bp, Borrelia garinii
gcaatctgagactagaaaag

SEQ ID NO: 27 - DNA, 20 bp, Borrelia garinii
tgcagtctttacacttactt

SEQ ID NO: 28 - DNA, 26 bp, Borrelia garinii
ccctcgagagtaccaaatgccattct

SEQ ID NO: 29 - DNA, 22 bp, Borrelia garinii
acatattatgtagcctgtttta

SEQ ID NO: 30 - DNA, 21 bp, Borrelia afzelii
ctttaaaggagagaaagcatg

SEQ ID NO: 31 - DNA, 31 bp, Borrelia afzelii
ccggatccgatttattcataagagatgaaat

SEQ ID NO: 32 - DNA, 20 bp, Borrelia afzelii
tgagcataaaaggatgcttc

SEQ ID NO: 33 - DNA, 18 bp, Borrelia afzelii
gcagtccttgcactcact

SEQ ID NO: 34 - DNA, 28 bp, Borrelia afzelii
ccctcgagcaaagattagtaccaaacac

SEQ ID NO: 35 - DNA, 22 bp, Borrelia afzelii
acatattatgtagcctgtttta

(19) United States
(12) Patent Application Publication
(10) Pub. No.: US 2006/0034862 A1
(43) Pub. Date: Feb. 16, 2006

(54) Method for diagnosing early and late lyme borreliosis

(76) Inventors: Pekka Lahdenne, Helsinki (FI); Tero Heikkila, Helsinki (FI); Ilkka Seppala, Helsinki (FI); Harri Saxen, Helsinki (FI)

Correspondence Address: BIRCH STEWART KOLASCH & BIRCH, PO BOX 747, FALLS CHURCH, VA, 22040-0747, US

(21) Appl. No.: 10/496,647

(22) PCT Filed: Nov. 26, 2002
(86) PCT No.: PCT/FI02/00951

§ 371 Date: Jul. 27, 2004

(30) Foreign Application Priority Data
Nov. 26, 2001 (FI) 20012310

(51) Int. Cl. G01N 33/554; A61K 39/02; G01N 33/569

(52) U.S. Cl. 424/190.1; 435/7.32

(57) ABSTRACT

The present invention relates to a method for detecting Borrelia burgdorferi sensu lato infection or the presence of antibodies against Borrelia species in a body fluid from a suspected infected or vaccinated human or other mammal, to a diagnostic kit useful in said method and to an immunoassay method for diagnosing early and late Lyme borreliosis, especially for diagnosing erythema migrans. The methods according to the invention are characterized in that recombinant BBK32 proteins, optionally other immunogenic borrelial proteins, or their fragments are used as antigens.

### FIELD OF THE INVENTION

The present invention relates to a method for detecting Borrelia burgdorferi sensu lato infection or the presence of antibodies against Borrelia species in a body fluid from a suspected infected or vaccinated human or other mammal, to a diagnostic kit useful in said method and to an immunoassay method for diagnosing early and late Lyme borreliosis (LB), especially for diagnosing erythema migrans (EM). The methods according to the invention are characterized in that recombinant BBK32 proteins, other immunogenic borrelial proteins and/or their fragments are used as antigens.

### BACKGROUND OF THE INVENTION

LB is a tick-transmitted spirochetal infectious disease, caused by Borrelia burgdorferi, which is characterized by multistage skin, joint, neurologic and cardiac manifestations [1]. The diagnosis of LB is based on clinical evaluation of the patients, but serologic assays, most frequently the enzyme-linked immunosorbent assay (ELISA) and Western blotting (WB), are often used to provide supporting evidence of infection with B. burgdorferi. In the current routine LB serodiagnostic tests, the antigens predominantly used are borrelial flagellin protein or whole-cell lysate (WCL) of the in vitro-cultured microbes. A two-step approach with ELISA and a confirmatory WCL WB has been recommended by the Centers for Disease Control (ISA) for positive or borderline results [2]. Especially in Europe the applicability of this procedure has remained questionable [3], mainly due to the existence of three species of B. burgdorferi sensu lato causing human LB, B. burgdorferi sensu stricto, B. afzelii, and B. garinii [4]. One of the main reasons for these problems is the antigenic diversity due to variations in the sequences and expression of immunogenic proteins in Several other difficulties complicate current LB serology. In LB patients with early manifestations, e.g. erythema migrans (EM) and facial palsy, antibody responses to the current antigens may be weak or delayed [6]. EM, which appears at the site of the tick bite days to weeks after exposure, is the earliest and most common manifestation of LB. Tick bites may easily be unrecognised, and the clinician has to rely on the appearance of the skin lesion. In a routine clinical setting, EM is considered to be pathognomonic for early LB. The classical appearance of EM is an enlarging, ring-like erythema with a central clearing. However, early in the course of LB, atypical lesions may occur and cause diagnostic problems. Recent reports from the US and Europe have shown that during the first few days of infection the pathognomonic expanding peripheral erythema with a central clearing appears to be far less common than lesions with homogeneous redness [33, 34]. The gold standard for the diagnosis of early LB is microbiologic confirmation of LB by culture from biopsies taken from EM lesions and/or from blood. PCR-based methods can also be used to detect B. burgdorferi DNA in skin biopsies. However, in routine clinical practice, these methods are not feasible. Serologic confirmation of early LB is also problematic due to sensitivity problems of the current serodiagnostic assays. The sensitivity of the IgM or IgG enzyme-linked immunosorbent assay (ELISA) using borrelial flagella or whole-cell lysate antigens seldom exceeds 40-50% [6, 25]. Even at late stage LB, up to 5-10% of patients may not have elevated antibody levels [7]. Further, viral infections cause false positivity in several LB tests for IgM antibodies [8]. Furthermore, in a subgroup of patients after a successful treatment of LB, antibody levels may stay high even for prolonged periods.

The expression of borrelial proteins also varies at different stages in the life cycle of Borrelia in ticks and in the mammalian hosts. Several genes, e.g. bbk32, bbk50, vls and ospE/F homologs [9-11], have been shown to be selectively expressed in vivo. Moreover, bbk32 expression is detectable in spirochetes during tick feeding even before transmission to the host but not in unfed ticks [12]. In two previous studies, antibodies to BBK32 were observed in the sera of B. burgdorferi sensu stricto-infected mice and human patients with disseminated LB [9, 13]. So far, antigenic properties of the BBK32 proteins in other species of B. burgdorferi sensu lato are not known.

The application of specific recombinant proteins of B. burgdorferi, such as outer surface protein A (OspA), OspB, OspC, OspE, OspF, P22, P39, P100, VlsE, and flagellin have improved the performance of ELISA assays [14-16]. However, the immune responses of patients infected with different B. burgdorferi subspecies vary greatly, and certain antigens may not always be expressed in hosts or be recognized immunologically. Thus, the sensitivity with single recombinant antigens has remained insufficient, so far. Another approach to improve the serodiagnosis of LB has been the use of peptides from certain borrelial proteins as antigens, e.g. a C-terminal de-capeptide from OspC [17] and a peptide corresponding to a central invariable region in the VIsE protein, invariable region 6, IR6 [18]. Very recently, chimeric proteins comprising of epitopes of OspA, OspB, OspC, flagellin, and p93 have also been suggested for serodiagnostic antigens [19].

In the literature, the BBK32 protein has also been called P35 [9, 13, 20] and 47 kilodalton fibronectin binding (FBP) protein [21].

### DETAILED DESCRIPTION OF THE INVENTION

The purpose of the inventors was to test the antigenic potential of borrelial protein BBK32 and develop a reliable immunoassay method for the serodiagnosis of early and disseminated LB, especially for the serodiagnosis of erythema migrans. In order to cover all pathogenic borrelial species causing human LB, the inventors sequenced and cloned the bbk32 from eight isolates of the three pathogenic borrelial species, B. burgdorferi sensu stricto, B. afzelii, and B. garinii. The identity between the amino acid sequences of BBK32s in B. burgdorferi sensu stricto, B. garinii, and B. afzelii isolates was 71-100%. The respective variant BBK32 recombinant proteins were tested in LB serology using serum samples from patients with early- and late-stage LB. In IgG Western blotting (WB) or enzyme-linked immunosorbent assay (ELISA), up to 74% and 100% of acute and convalescent samples from 23 patients with erythema migrans (EM) were positive for recombinant BBK32 protein from B. afzelii. In the serology of disseminated LB, the three variant BBK32 antigens cross-reacted. In total, the sensitivity of IgG ELISA reached 100%. The results show that the BBK32 proteins are useful serodiagnostic antigens for early and disseminated LB, but in order to cover all the relevant borrelial species variant BBK32 proteins or fragments thereof should be used in parallel or combined in an immunoassay for LB.

It is therefore an object of the present invention to provide a novel method for detecting Borrelia burgdorferi sensu lato infection or the presence of antibodies against Borrelia species in a body fluid from a suspected infected or vaccinated human or other mammal, in which method recombinant BBK32 proteins or their fragments containing antigenic epitopes from one or more Borrelia species are used as antigens in an immunoassay. The method according to the invention can be used for the serodiagnosis of early and late Lyme borreliosis, especially for the serodiagnosis of erythema migrans.

Within the scope of this invention and this specification, a ‘fragment’ is intended to mean a recombinantly produced fragment containing antigenic epitopes of the immunogenic protein(s) in question.

In a preferred embodiment according to the invention, recombinant BBK32 proteins or their fragments together with any other immunogenic proteins or their fragments derived from one or more Borrelia species are used as antigens in an immunoassay. Examples of the other immunogenic borrelial proteins which can be used in the method according to the invention include, but are not limited to, outer surface protein A (OspA), OspB, OspC, OspE, OspF, P22, P39, P100, VlsE, DbpA, and/or flagellin, preferably DbpA.

Preferably the recombinant BBK32 proteins, optional other immunogenic proteins, or their fragments are derived from at least two Borrelia species selected from the group consisting of B. burgdorferi sensu stricto, B. afzelii, and B. garninii, more preferably from all the three Borrelia species mentioned above.

It is apparent to those skilled in the art that instead of borrelial proteins or their fragments, or together with them, also peptides or polypeptides (referred here as peptides), i.e. shorter amino acid stretches comprising at least two, usually several amino acids, can be used as antigens in the method according to the invention. It is also possible to take a combination of several fragments or peptides from various immunogenic borrelial proteins and to use them as antigens.

The recombinant BBK32 proteins, optional other immunogenic proteins, or their fragments are used as antigens either in parallel or combined in an immunoassay. When recombinant BBK32 proteins are used as ‘parallel’ antigens, antibodies are measured separately against the recombinant BBK32 antigens from different Borrelia species in the same assay. It is also possible to combine the recombinant BBK32, other immunogenic borrelial antigens, or their fragments from different Borrelia species and measure antibodies against the ‘combined’ BBK32 antigen. For example in a parallel assay, three recombinant BBK32 protein antigens from three Borrelia species are preferably used in the same assay. In a combined assay, preferably three recombinant BBK32 proteins from three Borrelia species are combined to form the combined BBK32 protein antigen.

Within the scope of this invention, an ‘immunoassay’ is intended to cover all immunoassay methods known to persons skilled in the art.

The method according to the invention for detecting Borrelia burgdorferi sensu lato infection or the presence of antibodies against Borrelia species in a body fluid from a suspected infected or vaccinated human or other mammal comprises preferably the steps of
a) contacting the body fluid with recombinant BBK32 proteins, optional other immunogenic proteins, or their fragments derived from one or more Borrelia species under conditions effective to allow the formation of antigen-antibody complexes; and
b) detecting the complexes formed.

The body fluid is preferably a serum, plasma, whole blood, cerebrospinal fluid, or synovial fluid sample.

The conditions effective to allow the formation of antigen-antibody complexes as well as the means for detecting the complexes formed are chosen according to the antibodies and other reagents used in the assay and are known to a person skilled in the art.

It is a further object of the invention to provide a diagnostic kit useful for detecting Borrelia burgdorferi sensu lato infection or the presence of antibodies against Borrelia species in a body fluid from a suspected infected or vaccinated human or other mammal, said kit comprising in a suitable container
a) recombinant BBK32 proteins, optional other immunogenic proteins, or their fragments from one or more Borrelia species and a detectable label or marker linked to said proteins and/or their fragments, or
b) recombinant BBK32 proteins, optional other immunogenic proteins, or their fragments from one or more Borrelia species and a second antibody linked to any detectable label or marker.

Detectable labels or markers and methods to link them to antigens or second antibodies are well disclosed in the literature and are also known to persons skilled in the art of immunoassays.

A further object of the invention is an immunoassay method for diagnosing early and late Lyme borreliosis, especially for the serodiagnosis of erythema migrans, comprising the steps of
a) contacting a body fluid from a human or other mammal with recombinant BBK32 proteins, optional other immunogenic proteins, or their fragments derived from one or more Borrelia species under conditions effective to allow the formation of antigen-antibody complexes; and
b) detecting the complexes formed.

A still further object of the invention is to provide a novel method for detecting Borrelia burgdorferi sensu lato infection or the presence of antibodies against Borrelia species in a body fluid from a suspected infected or vaccinated human or other mammal, in which method recombinant BBK32 proteins and recombinant decorin binding protein As (DbpAs) from at least two, preferably three, Borrelia species are used together as antigens in an immunoassay. Decorin binding protein A is a borrelial outer surface protein, which has been suggested to act as a species-specific serodiagnostic antigen for LB. In a preferred embodiment according to the invention, the recombinant BBK32 proteins and the recombinant DbpAs are derived from B. burgdorferi sensu stricto, B. afzelii, and B. garinii.

Antibodies to the BBK32 protein seem to appear very early during human LB. In up to 74% of the patients at acute-phase EM, IgG antibodies to BBK32 were detectable by ELISA and/or WB. At follow-up, after successful antibiotic treatment, all the patients were anti-BBK32 antibody-positive. A recent study reported cloning of the bbk32 from a B. burgdorferi sensu stricto isolate and showed early antibody responses to the recombinant BBK32 protein during experimental murine borreliosis [9]. Reverse transcriptase-PCR studies have also demonstrated bbk32 expression in EM lesions of three patients, indicating that, during human LB, bbk32 is expressed early [20]. However, antibody responses to BBK32 in patients with early local EM have not previously been studied.

With the current LB serology based mainly on flagellin as an antigen, IgM and IgG antibodies are not detectable by ELISA or immunoblot assays until 2-4 or 6-8 weeks after the onset of the disease [6]. During early local LB, the sensitivity of IgM ELISA seldom exceeds 50% [6, 22-24]. A study on patients with culture-confirmed EM showed that positive serology at presentation and the rate of seroconversion correlated directly with disease duration [25]. If the EM lesion had emerged less than 7 days prior to sampling, only 10% of the patients showed antibodies in ELISA, whereas, of the patients whose EM had occurred 7 to 14 days earlier, 58% had detectable antibodies. A recent study reporting on anti-BBK32 antibodies in disseminated LB also showed IgG BBK32 antibodies in 84% of patients with EM [13]. However, in these patients, the EM lesion had been present for 2 weeks to 3 months after disease onset, suggesting dissemination of LB. In the present series, the time of occurrence of the EM lesion could not be accurately assessed. However, given the low proportion of seropositivity to flagellin at presentation of EM (17-26%) and the broad awareness of LB among the general population in regions where LB is endemic in Finland, it can be presumed that, in most cases, the EM lesions represented early disease. Hence, the present results imply that assessment of IgG, although not of IgM antibodies, to BBK32 proteins affords a major improvement in the serodiagnosis of early LB. The antibody response to the BBK32 protein also seems to precede the humoral response to in vitro-grown microbes (WCL), which do not necessarily synthesize this in vivo-expressed protein [9, 21].

Only a few studies have evaluated the antigenic properties of the BBK32 protein. In these studies, the BBK32 proteins originated from American B. burgdorferi sensu stricto strains [13, 20]. In a preferred embodiment of the present invention variant recombinant proteins from the three pathogenic borrelial species, B. burgdorferi sensu stricto, B. afzelii, and B. garinii, are used in the serodiagnosis of LB, especially in the serodiagnosis of erythema migrans. Recombinant BBK32 originating from a local B. afzelii isolate appeared to be superior to the other rBBK32 proteins for diagnosing EM. This finding agrees with the PCR results of the EM skin biopsies, where the majority of the infecting species proved to be B. afzelii. These observations are also in accord with two recent European studies, where over 90% of Borrelia isolates from EM lesions were B. afzelii, less than 10% B. garinii, and none were B. burgdorferi sensu stricto [26, 27]. The present results suggest that in diverse epidemiological situations, for the serodiagnosis of early local EM, variant BBK32 proteins are preferred.

Although the immunoreactivity of EM patient sera to variant BBK32 proteins diverged, the three recombinant BBK32 antigens “cross-reacted” in the serology of disseminated LB. Furthermore, the intensities of the serologic responses against variant BBK32 proteins, as measured by OD values in ELISA, correlated well. Therefore, these results indicate that variant BBK32 proteins may have both specific and common antigenic epitopes. In European epidemiological studies, the most prevalent Borrelia species have been B. afzelii and B. garinii [28], B. burgdorferi sensu stricto occurring infrequently, especially in Scandinavia [27, 29]. In cerebrospinal fluid samples of European neuroborreliosis cases, the species predominantly isolated has been B. garinii [30]. The hypothesis that epitope specificity varies in early and late LB is in line with the analysis of BBK32 sequences of eight Finnish isolates of B. burgdorferi sensu lato, showing over 90% identity between the sequences of B. burgdorferi sensu stricto and B. garinii. In contrast, the identity between the BBK32 sequences of B. afzelii strains and of other species was approximately 70%.

In the serodiagnosis of disseminated LB, the BBK32 antigen has been evaluated in a limited number of patients only, so far [9, 13]. Fikrig et al. [9] reported high IgG antibodies to BBK32 in 3 of 3 patients with neuroborreliosis and in 3 of 7 patients with Lyme arthritis. Akin et al. [13] showed IgG responses to BBK32 in 83-92% of 25 Lyme arthritis patients. The preferred embodiment of the present invention with the three variant BBK32 proteins as antigens improved the sensitivity of ELISA up to 100%. In the serodiagnosis of neuroborreliosis, all but one case would have been detected irrespective of the origin of the BBK32 antigen. Instead, especially in ELISA for Lyme arthritis, use of a single BBK32 antigen would have left the sensitivity at 80-90%. The occasional discrepancies between the results of WB and ELISA may be due to differences in the orientation of the antigen and/or antigen-antibody complex formation.

In summary, the inventors have shown that the BBK32 proteins are useful antigens for both early and late LB serology. The BBK32 from B. afzelii proved to be a sensitive antigen of EM already at presentation. During the course of infection, the sensitivity increased being up to 100% in convalescence samples for EM patients. However, it is evident that variant BBK32 proteins should be used either in parallel or combined with an immunoassay for LB to cover all the relevant borrelial species, whose prevalence differs regionally in Europe.

The following examples further illustrate the invention without, however, limiting the same.

### EXAMPLES

Bacterial strains. Finnish borrelial strains were obtained from the National Public Health Institute, Turku, Finland. B. burgdorferi sensu stricto strain ia (here referred to as Bbia) was isolated from the cerebrospinal fluid of a Finnish patient with neuroborreliosis. Of the B. afzelii strains, A91 and 1082 (referred to as BaA91 and Ba1082) were isolated from skin biopsy samples of Finnish patients with erythema migrans EM), and 570 and 600 (referred to as Ba570 and Ba600) were isolated from ticks. B. garinii strains 40, 46, and 50 (referred to as Bg40, Bg46, and Bg50, respectively) were isolated from skin biopsy samples of Finnish patients with EM. The genotypes of culture-positive Borreliae were confirmed by sequencing a fragment of the flagellin gene [29]. Borreliae were cultivated in BSK-H (Barbour-Stoenner-Kelly) medium (Sigma, USA) at 33° C. in 5% CO₂. The B. afzelii strain SK1 was used in an in-house ELISA for detecting antibodies against borrelial WCL. Escherichia coli host cells for cloning and for expression of recombinant proteins were INFαF (Invitrogen, Netherlands) and BL21 (Amersham Pharmacia Biotech, Sweden), respectively.

DNA purification. Borrelial genomic DNA was purified with a Dneasy Tissue Kit (Qiagen, Germany). Purified DNA was used in PCR and in cloning experiments. Plasmid DNA was purified with a QIAprep-spin plasmid kit (Qiagen, USA).

PCR and DNA sequencing. A PCR-based approach was employed to amplify and sequence the bbk32 alleles from eight different isolates of B. burgdorferi sensu lato. Primers for bbk32 sequencing were designed on the basis of published bbk32 sequences (Table 1). Several primer pairs were designed and tested to ensure that the entire coding sequence of the bbk32 was obtained. To eliminate possible errors caused by Taq-polymerase, the two strands for each bbk32 were sequenced independently at least twice. Expression primers for each strain encoding the mature portion of the BBK32 protein after cysteine at the site of posttranslational acylation were chosen from the sequences analyzed. For each borrelial strain, the bbk32 sequences were generated by PCR amplification of B. burgdorferi genomic DNA. Approximately 1 ng of template DNA was used in standard PCR conditions: 30 cycles of 94° C. denaturing for 1 min, 50° C. annealing for 1 min, and 72° C. extension for 1 min 30 s with AmpliTaqGold DNA polymerase (Perkin Elmer, USA). The PCR-amplified full-length or partial bbk32s were cloned to the pCR 2.1-TOPO vector (Invitrogen, Netherlands) for sequencing. DNA sequencing was performed at the Core Facility of the Haartman Institute, University of Helsinki, with DyePrimer (T7, M13Rev) cycle sequencing kit (Applied Biosystems Inc., USA). Sequencing reactions were run and analyzed by the automated sequencing apparatus model 373A (Applied Biosystems Inc., USA). DNA and protein sequences were analyzed with Lasergene software (DNASTAR, USA).

TABLE 1

Primers used for PCR amplification of the bbk32 genes

| | | | | |
|---|---|---|---|---|
| No. | Species | Primer 5′-3′ | Location | Source |

| | | | | |
|---|---|---|---|---|
| 1 | B. buredorferi | CAC CCT CTT GAT AGC ACT TA | −203--184 | B31(AF000788) |
| 2 | sensu stricto | CTT TAA AGG AGA GAA AGC ATG | −18-3 | Bbia(AF472532) |
| 3 | | CCG GAT CCG ATT TAT TCA TAA GAT ATG AAA T | 60-82 | Bbia |
| 4 | | GCA ATC TGA GAC TAG AAA AG | 329-348 | Bbia |
| 5 | | TGC AGT CTT TAC ACT TAC TT | 879-860 | Bbia |
| 6 | | CCC TCG AGA TTA GTA CCA AAC GCC ATT | 1084-1065 | Bbia |
| 7 | | ACA TAT TAT GTA GCC TGT TTT A | 1122-1101 | B31 |
| 2 | B. garinii | CTT TAA AGG AGA GAA AGC ATG | −18-3 | Bbia |
| 3 | | CCG GAT CCG ATT TAT TCA TAA GAT ATG AAA T | 60-82 | Bg40(AF472529) |
| 4 | | GCA ATC TGA GAC TAG AAA AG | 329-348 | Bg40 |
| 5 | | TGC AGT CTT TAC ACT TAC TT | 879-860 | Bg40 |
| 8 | | CCC TCG AGA GTA CCA AAT GCC ATT CT | 1084-1064 | Bg40 |
| 7 | | ACA TAT TAT GTA GCC TGT TTT A | 1122-1101 | B31 |
| 2 | B. afzelii | CTT TAA AGG AGA GAA AGC ATG | −18-3 | Bbia |
| 9 | | CCG GAT CCG ATT TAT TCA TAA GAG ATG AAA T | 57-79 | BaA91(AF472525) |
| 10 | | TGA GCA TAA AAG GAT GCT TC | 369-387 | BaA91 |
| 11 | | GCA GTC CTT GCA CTC ACT | 855-838 | BaA91 |
| 12 | | CCC TCG AGC AAA GAT TAG TAC CAA ACA C | 1065-1046 | BaA91 |
| 7 | | ACA TAT TAT GTA GCC TGT TTT A | 1122-1101 | B31 |

Restriction enzyme sites for BamHI and XhoI in expression primers are underlined
Primers 2 and 7 were used in all strains
Primers 3, 4, and 5 were used both in B. burgdorferi sensu stricto and B. garinii PCR amplifications

Cloning and expression of recombinant BBK32. For expression of the recombinant BBK32 (rBBK32), glutathione S-transferase (GST) fusion protein constructs were generated. The PCR-amplified DNA encoding the mature portion of BBK32 was cloned into the pCR 2.1-TOPO plasmid (Invitrogen, Netherlands). The recombinant plasmid was purified and digested with BamHI and XhoI restriction enzymes. The cleaved bbk32 was then ligated to a similarly digested pGEX4T-1 expression plasmid (Amersham Pharmacia Biotech, Sweden) and transformed into E. coli BL21 host cells. The expression of recombinant GST-BBK32 protein was generated according to the manufacturer's instructions (Amersham Pharmacia Biotech, Sweden). The expression and purity of the GST-rBBK32 fusion protein was confirmed by sodium dodecyl sulfate-polyacrylamide gel electrophoresis (SDS-PAGE).

Western blotting. GST-rBBK32s originating from Bbia, BaA91, and Bg40 (referred to here as rBBK32_{Bbia}, rBBK32_{BaA91}, or rBBK32_{Bg40}, respectively) were fractionated in 10% SDS-PAGE and transferred to a nitrocellulose membrane (BioRad, 0.2 μm pore size, USA) by semi-dry transfer with 40 mM glycine-50 mM Tris (pH 9.0)-0.375% (w/v) SDS-20% (v/v) methanol buffer. Equal amounts of each GST-rBBK32 were used for one 7-cm-wide nitrocellulose membrane. Two-mm strips of the nitrocellulose membranes were soaked in 0.1% Tween 20, 0.9% NaCl. Serum samples were diluted in 0.1% Tween 20, 0.9% NaCl, 0.1 g/l fat-free bovine milk powder (Valio, Finland). Samples were incubated at 1:100 dilution for 2 h. After four buffer rinses, the Western blots were incubated with alkaline phosphatase-conjugated rabbit anti-human IgG (Jackson Immuno Research Laboratories Inc., USA) at 1:5000 for 2 h. After washing, the bands were visualized with 5-bromo-4-chloro-3-indolylphosphate nitro blue tetrazolium (Sigma Chemical Co., USA). The reaction was terminated 10-15 min later by washing with distilled water. The WB results were analyzed with MacBAS 2.5 (Fuji, Japan) software, and the cut-off for a positive IgG WB result was defined as the mean+3 standard deviations (SD) of the values of healthy blood donors. For detection of GST, monoclonal anti-GST antibodies (Sigma, USA) were used.

ELISA. ELISA analyses for anti-flagellin antibodies were done as described earlier [31]. Briefly, IgG antibodies against B. burgdorferi were measured with a commercial flagellin-based ELISA kit (Dako, Denmark) modified by titrating the antibodies. Sera were diluted serially in three-fold steps for the test and applied to the plates for overnight incubation. The bound antibodies were detected with biotin-labeled goat anti-human IgG (Zymed, USA). An end-point titer was obtained at an optical density level determined by a cut-off control provided by the kit. The titer limit for a positive IgG antibody level was 500. The cut-off control material conformed with the level of the mean+3 SD of the reference population living in central Finland, an area with low prevalence of LB [31].

For ELISA assays measuring anti-BBK32 antibodies, the wells in a microtiter plate were coated with 100 μl (2 μg/ml) of variant BBK32 recombinant proteins overnight. After washing, 100 μl of diluted serum samples were added to the wells and incubated overnight. Serum samples were diluted 1:10 (EM) or 1:100 (neuroborreliosis and Lyme arthritis) in 5 mg/ml bovine serum albumin (BSA) in 0.155 M NaCl-0.04% Tween 20 buffer (BSA-NaCl-Tween). After washing, the wells were incubated with alkaline phosphatase-conjugated rabbit anti-human IgG or IgM (Jackson Immuno Research Laboratories Inc., USA) 1:5000 in BSA-NaCl-Tween for 2 h. The reactions were visualized with 4-nitrophenylphosphate (Boehringen Mannheim GmbH, Germany) 1 mg/ml in diethanolamine buffer pH 10.0. The optical density (OD) measurements were made after 10 to 20 minutes at wavelength 405 nm using a Multiscan photometer (Thermo Labsystems, Finland).

Samples. 1. For serological analyses, human serum samples were collected from Finnish patients with culture- or PCR-positive EM, with neuroborreliosis (NB), and with Lyme arthritis (LA). Samples were collected from EM patients at diagnosis (acute) and 1 to 3 months after treatment (convalescent). Of the 23 patients with EM, genotyping by PCR analysis [29] showed B. afzelii in 17, and B. garinii in 4 of the skin biopsies. In two biopsies, genotyping was not feasible. In the patients with disseminated LB, the clinical manifestations agreed with the CDC criteria for LB [32]. The clinical diagnosis was confirmed in ELISA by demonstrating serum antibodies (and CSF anti-flagellin antibodies in NB patients) against flagellin and B. burgdorferi WCL. Serum samples from patients with syphilis, Epstein-Barr virus (EBV) infection, systemic lupus erythematosus (SLE), rheumatoid factor (RF) positivity, anti-streptolysin (ASO) positivity, and sera from healthy blood donors were used as controls.

2. Serum samples were also collected from patients with clinically documented or culture- or PCR-confirmed EM from Germany, Slovenia, and USA. German samples were collected in Northern Bavaria as part of a regional study on LB [35]. Sera were collected from 22 patients with physician-diagnosed EM at the time of diagnosis and during the convalescence phase. The clinical diagnosis of EM was confirmed by especially trained physicians. In addition, from 10 US patients with culture-positive EM sera were collected at the time of diagnosis and during convalescence 3-4 weeks after treatment [36]. Serum samples were available from 20 Slovenian patients in the acute stage of EM. Two out of 9 Slovenian patients from whom skin biopsies were taken were culture-positive (B. burgdorferi sensu lato) [37]. All patients with EM were treated with oral antimicrobials.

Serum samples from 40 Finnish healthy blood donors were used as negative controls and to define the cut-off value for all ELISAs (cut-off=mean plus 3 SD).

Nucleotide sequence accession numbers. The nucleotide sequences of the bbk32 were submitted to GenBank under accession numbers AF472525 for B. afzelii A91 (SEQ ID NO. 1), AF472527 for B. afzelii 1082 (SEQ ID NO. 5), AF472526 for B. afzelii 570 (SEQ ID NO. 3), AF472528 for B. afzelii 600 (SEQ ID NO. 7), AF472529 for B. garinii 40 (SEQ ID NO. 9), AF472530 for B. garinii 46 (SEQ ID NO. 11), AF472531 for B. garinii 50 (SEQ ID NO. 13), and AF472532 for B. burgdorferi sensu stricto ia (SEQ ID NO. 15).

Statistical analyses. The Microsoft Excel 2000 program (Microsoft, USA) was used for calculations of standard statistics.

Results

Sequence analysis of BBK32 in the Finnish borrelial isolates. The deduced amino acid sequences of BBK32_{BaA91}, BBK32_{Ba1082}, BBK32_{Ba570}, BBK32_{Ba600}, BBK32_{Bg40}, BBK32_{Bg46}, BBK32_{Bg50}, and BBK32_{Bbia} contained 352 to 360 residues. The sequences of all the BBK32 proteins revealed a hydrophobic leader sequence of 18 to 19 residues and a phenylalanine-X-Y-cysteine motif, consistent with a lipoprotein. Characteristically for borrelial lipoproteins, the greater part of the mature portion of the BBK32 protein was hydrophilic (data not shown). The BBK32 leader sequences in the B. garinii strains and the B. burgdorferi sensu stricto strain were identical, but differed by three amino acids from the leader sequences in the identical B. afzelii strains. The inter-species identity of the deduced amino acid sequences of the BBK32 proteins ranged from 71 to 95% (FIG. 1). The differences in the amino acid sequences were distributed evenly along the sequence. The identity of the BBK32 amino acid sequences within the borrelial subspecies ranged from 94 to 100%. The calculated molecular mass of the mature BBK32 proteins (without putative lipid acylation) ranged from 38.7 to 39.5 kDa.

Sequence analysis of BBK32 in B. afzelii strains. The inventors sequenced the bbk32 from two human (BaA91 and Ba1082) and two tick isolates (Ba570 and Ba600). The identity of the deduced amino acid sequences was from 99 to 100%. The BBK32 sequences of the two human B. afzelii isolates were identical. In the GenBank search, one bbk32 sequence from B. afzelii was found. The BBK32 sequence of the ACA1 strain (AF213179) is only partial, corresponding to the sequence between amino acid positions 34 and 336 of the deduced sequence of BBK32_{BaA91}. In the matching regions of the four studied BBK32 sequences from B. afzelii and the partial BBK32_{ACA1}, sequence, identity was from 99 to 100%.

Sequence analysis of BBK32 in B. garinii strains. The deduced amino acid sequences of BBK32_{Bg40} and BBK32_{Bg50} were identical, and BBK32_{Bg46} was 94% identical with them. In the GenBank search, one partial bbk32 sequence from B. garinii strain Ip90 (AF213178) was found. This sequence matched the BBK32_{Bg40} sequence between amino acid residues 35 and 343, but in the BBK32_{Ip90} sequence a six amino acid deletion was observed from positions 201 to 206. Sequence identity in the corresponding regions of the BBK32 sequences from B. garinii strains in this study and the partial BBK32_{Ip90} sequence was from 92 to 93%.

Sequence analysis of BBK32 in B. burgdorferi sensu stricto strains. The bbk32 sequence from the local strain Bbia was compared with two sequences published in the GenBank. The bbk32 sequence of the B31 strain (AE000788) was complete and that of strain N40 (U82107) was a partial sequence, lacking the first 86 amino acids. The BBK32 amino acid sequences from Bbia and B31 were 96% identical. Identity in the corresponding regions of BBK32 sequences of Bbia, B31, and N40 was from 91 to 94%. In the BBK32_{B31} sequence there was a six amino acid deletion at positions 201 to 206 of BBK32_{Bbia}. In the same region, three tyrosine residues were deleted from the BBK32_{N40} sequence.

Western blotting. In IgG Western blots using rBBK32 proteins from B. afzelii (BaA91) as antigen with serum samples from Finnish patients with culture- or PCR-positive EM, 10/15 (67%) at the acute and 15/15 (100%) at the convalescent phase were positive FIG. 2). With rBBK32_{Bg40} as an antigen, 4/15 and 8/15, and with rBBK32_{Bbia}, 7/15 and 3/15 were positive at the acute and convalescent phases, respectively. Of the 10 control patients (five with syphilis and five healthy blood donors), one with syphilis had a weak antibody response to rBBK32_{BaA91} (data not shown). In ELISA for anti-fiagellin antibodies (Dako, Denmark), 6/15 patients had IgG or IgM antibodies at the acute or convalescent phase (FIG. 2). One patient had IgM antibodies in the in-house ELISA (WCL as antigen).

In another WB experiment, serum samples from 10 patients with NB and 10 with LA were analyzed for the presence of anti-BBK32 IgG antibodies. All 20 patients with disseminated LB reacted positively with the three rBBK32 proteins as determined with the MacBAS program, using the blood donors' mean+3 SD as a cut-off value (Table 2). Minor differences were observed in the immunoreactivity of serum samples against different rBBK32 proteins. Specifically, in four serum samples, weaker reactivities were observed against rBBK32_{BaA91} than against the other rBBK32s (data not shown). A few of the control samples were low positives (Table 2). None of the patient or control sera recognized pure GST (data not shown).

TABLE 2
IgG Western blotting reactivity against recombinant BBK32 proteins
from B. afzelii (BaA91), B. garinii (Bg40), and B. burgdorferi
sensu stricto (Bbia) of LB patients and controls. The intensities
of WB bands were analyzed with MacBas 2.5 software.

| | No. of positive samples/ | | |
|---|---|---|---|
| Patient/ | no. of samples recombinant BBK32 | | |
| control group | BaA91 | Bg40 | Bbia |
| Neuroborreliosis | 10/10 | 10/10 | 10/10 |
| Lyme arthritis | 10/10 | 10/10 | 10/10 |
| Syphilis | 1/5 | 0/5 | 2/5 |
| Positive for RF* | 0/5 | 0/5 | 1/5 |
| Blood donors | 0/5 | 0/5 | 0/5 |

*RF, rheumatoid factor

ELISA. 1. Serum samples from Finnish LB patients and controls were tested in IgG ELISA, using all three rBBK32 proteins individually as antigens. In patients with NB, 14/14, 13/14, and 14/14 samples were positive when rBBK32_{BaA91}, rBBK32Bg₄₀, and rBBK32_{Bbia}, respectively, were used as antigens. In serum samples from patients with LA, these figures were 12/15, 11/15, and 14/15, respectively. In total, 14 of 14 (100%) samples from patients with NB, and 15 of 15 (100%) samples from patients with LA were positive for one or more rBBK32 proteins (FIG. 3). The ELISA OD values of NB patients correlated well between variant BBK32 proteins as antigens, the correlation coefficients being 0.91, 0.78, and 0.83, between BBK32 from BaA91 and Bg40, BaA91 and Bbia, and Bg40 and Bbia, respectively. In LA patients, the respective correlation coefficients were 0.89, 0.90, and 0.93.

Serum samples from patients with EM were analyzed in both IgG and IgM ELISA. In IgG ELISA with rBBK32_{BaA91} as an antigen, 17/23 (74%) samples taken at the acute and 15/23 (65%) at the convalescent phase were positive (FIG. 4). On the other hand, when rBBK32Bg40 and rBBK32_{Bbia} were used as antigens, 6/23 (26%) and 5/23 (22%) of acute samples, and 7/23 (30%), and 4/23 (17%) of convalescent samples, respectively, were positive. Of the control samples, 2/10 with syphilis, 2/8 RF-positive, 4/10 EBV-positive, and 2/20 healthy blood donor samples showed low positive OD values. In IgM ELISA with samples from EM patients, 4 to 13% of the acute or convalescent samples were positive, depending on the rBBK32 antigen used.

In the ELISA tests for anti-flagellin antibodies, 6/23 (26%) patients had IgG antibodies in either acute or convalescent samples. In IgM ELISA assays for anti-flagellin antibodies, 4/23 (17%) were positive.

2. Antibodies in EM patients from different countries. At presentation of EM, 65 of the 75 (87%) patients Finnish, German, Slovenian, and American patients included) had IgG antibodies to one or more variants of rBBK32, 29/75 (39%) to flagella, and 29/75 (39%) to the IR₆ peptide antigen (Table 3, FIG. 5). In samples from different regions, reactivity to the rBBK32 variants diverged. The majority of patients from Finland had antibodies to rBBK32 from B. afzelii, whereas, in the German and Slovenian sera, the most sensitive antigen was rBBK32 from B. garinii. All 10 samples from the USA reacted positively with BBK32 from B. burgdorferi sensu stricto and from B. garinii.

In the different regions, the proportion of patients with IgM anti-flagella antibodies at the time of diagnosis varied from 13% to 45%. Of the total of 75 patients, 29% had IgM anti-flagella antibodies. IgM and/or IgG anti-flagella antibodies were detected in 46% of the patients.

Antibodies in the convalescent phase of EM patients. Forty of the 55 patients (73%) in the convalescent phase had IgG antibodies to one or more rBBK32s, 25/55 (45%) to flagella and 19/55 (35%) to the IR₆ peptide. The pattern of seropositivity to variant rBBK32s in the convalescent and the acute sera was similar. In the convalescence samples from Finland and Germany, the overall rate of IgG antibody positivity

BBK32 and from that at diagnosis (Table 3, FIG. 5).

The study with sera from epidemiologically diverse regions provides supporting evidence that the BBK32 proteins may be useful antigens in EM serology. At presentation of EM, the sensitivity of the BBK32 ELISAs appeared better than the anti-flagella or the new anti-IR₆ tests.

TABLE 3
Number of positive sera (%) for any of the three BBK32 variant antigens (BBK32 - all), IR₆ peptide antigen, and
for flagella antigen (Dako) in patients with EM from Finland (FIN), Germany (GER), USA, and Slovenia (SLO).

| | AT DIAGNOSIS | | | | | | | | | AT CONVALESCENCE | | | | | | |
|---|---|---|---|---|---|---|---|---|---|---|---|---|---|---|---|---|
| | FIN | | GER | | USA | | SLO | | TOTAL | FIN | | GER | | USA | | TOTAL |
| Antigen | n = 23 | | n = 22 | | n = 10 | | n = 20 | | n = 75 | n = 23 | | n = 22 | | n = 10 | | n = 55 |
| BBK32 - all* | 17 | (74%) | 21 | (95%) | 10 | (100%) | 17 | (82%) | 65 (87%) | 15 | (65%) | 15 | (68%) | 10 | (100%) | 40 (73%) |
| IR6* | 7 | (30%) | 7 | (32%) | 4 | (40%) | 11 | (55%) | 29 (39%) | 6 | (26%) | 6 | (27%) | 7 | (70%) | 19 (35%) |
| Flagella* | 6 | (26%) | 9 | (41%) | 4 | (40%) | 10 | (50%) | 29 (39%) | 6 | (26%) | 11 | (50%) | 8 | (80%) | 25 (45%) |
| Flagella** | 3 | (13%) | 8 | (36%) | 2 | (20%) | 9 | (45%) | 22 (29%) | 4 | (17%) | 7 | (32%) | 8 | (80%) | 19 (34%) |
| Flagella*** | 6 | (26%) | 13 | (59%) | 4 | (40%) | 12 | (60%) | 35 (46%) | 8 | (35%) | 15 | (68%) | 10 | (80%) | 33 (60%) |

*IgG class antibodies;
**IgM class antibodies;
***IgG and/or IgM antibodies

Antibodies to BBK32 in cerebrospinal fluid (CSF) samples. Antibodies to BBK32 have also been measured in the cerebrospinal fluid (CSF). CSF samples were obtained from 85 patients who had been treated for neuroborreliosis. The clinical diagnosis of neuroborreliosis was based on the clinical guidelines for diagnosis presented by the Centers for Disease Control and Prevention, USA. As a control assay, CSF antibodies to purified intact flagella of B. afzelii (Dako, Denmark) were determined. The CSF samples had also been studied for pleocytosis. Based on the anti-flagella antibodies and CSF findings, the patients were divided into three groups: confirmed (elevated anti-flagella IgG antibodies and pleocytosis in CSF), probable (elevated anti-flagella IgG antibodies but no pleocytosis in CSF), and possible (no anti-flagella IgG antibodies in CSF but either serum anti-flagella antibodies or pleocytosis in CSF) neuroborreliosis. In the classification, CSF anti-flagella antibodies were defined as significant if the IgG anti-flagella antibody titer in the CSF was higher than that of the serum divided by 400 (the approximate serum IgG/CSF IgG ratio in healthy persons). According to this definition, CSF anti-flagella antibody titers >20 were regarded as significant. Furthermore, patients were defined to have neuroborreliosis of short duration if the symptoms had lasted less than 3 months and of long duration if the symptoms had lasted longer than 3 months. As controls, we used CSF samples from 14 patients with syphilis, from 32 patients with other neurological diseases such as confirmed viral meningitis or convulsions/epilepsy, and from 20 patients without any proven infection. All the CSF samples from the-controls were negative for anti-flagella antibodies.

IgG ELISAs were performed with rBBK32 antigens, including three variants originating from B. burgdorferi sensu stricto, B. afzelii, and B. garinii, as described above. For rBBK32 ELISA, each well was coated overnight at +4° C. with 200 ng of protein, rBBK32 diluted in 3 M urea. The CSF samples were used at 1:10 dilution in NaCl-0.04% Tween 20 buffer (BNT). Samples of 20 CSF controls without proven infection were used to define the cutoff value (mean plus 3 SD).

Of all the 85 patients, 70% had CSF antibodies to rBBK32, whereas anti-flagella antibodies were observed in 53% of the patients. Of the 40 patients with negative or borderline anti-flagella antibodies (possible neuroborreliosis), 43% had anti-BBK32 antibodies (FIG. 6).

Of the 60 patients with duration of the disease <3 months, 73% had CSF antibodies to rBBK32. Of the 25 patients with disease of longer duration (>3 months), 58% had antibodies to rBBK32, mostly at low level (FIG. 7). In the patients with a longer duration of the disease, the antibody levels to rBBK32 in the CSF were lower than in the patients with a short duration (p<0.012) (FIG. 7).

Recombinant BBK32 fragment as an antigen. In another experiment, a 91-amino acid hydrophilic fragment of the BBK32 protein (from B. afzelii) (fragment) was initially cloned and expressed as a recombinant fusion protein (GST-fusion protein), purified and subsequently tested as an antigen in ELISA. As a comparison, antibodies to the BBK32 whole protein (GST-fusion protein) and to a commercial whole cell lysate antigen (Institut Virion/Serion GmbH, Germany) were assessed. The serum samples were from culture- or PCR-positive EM patients (n=23) or from patients with Lyme arthritis (n=7), neuroborreliosis (n=7) or acrodermatitis chronica atrophicans (n=3) and from control patients (syphilis, rheumatoid factor positivepatients, salmonella-, yersinia-infection, anti-streptolysin positive patients). In the serology of disseminated borreliosis, the performance of the BBK32 fragment in ELISA was as good as the BBK32 whole protein and whole cell lysate antigen but in the serology of erythema migrans, ELISA with the BBK32 fragment performed better than the control assays (Table 4).

TABLE 4

| | | IgG | IgG | Virion, | Virion, |
|---|---|---|---|---|---|
| | | BBK32 | fragment | IgG | IgM |
| EM I (n = 23) | Positive | 4 | 10 | 5 | 7 |
| | Borderline | | | 3 | 5 |
| | Negative | 19 | 13 | 15 | 11 |
| EM II (n = 23) | Positive | 6 | 12 | 5 | 6 |
| | Borderline | | | 3 | 6 |
| | Negative | 17 | 11 | 15 | 11 |
| Syfilis (n = 5) | Positive | 1 | 1 | 3 | 1 |
| | Borderline | | 1 | | 2 |
| | Negative | 4 | 3 | 2 | 2 |
| RF+ (n = 5) | Positive | 1 | 2 | 2 | 1 |
| | Borderline | | | | 1 |
| | Negative | 4 | 3 | 3 | 3 |
| Salmonella | Positive | | | | |
| (n = 5) | Borderline | | | | 1 |
| | Negative | 5 | 5 | 5 | 4 |
| Yersinia | Positive | | 1 | | 1 |
| (n = 5) | Borderline | | | 1 | |
| | Negative | 5 | 4 | 4 | 4 |
| AST+ (n = 5) | Positive | 1 | | | |
| | Borderline | | | 1 | 1 |
| | Negative | 4 | 5 | 4 | 4 |
| ACA (n = 3) | Positive | 3 | 3 | 3 | 2 |
| | Borderline | | | | 1 |
| | Negative | | | | |
| NB (n = 7) | Positive | 6 | 5 | 6 | 1 |
| | Borderline | | | | 4 |
| | Negative | 1 | 2 | 1 | 2 |
| LA (n = 7) | Positive | 7 | 6 | 7 | 4 |
| | Borderline | | | | 1 |
| | Negative | | 1 | | 2 |

### BRIEF DESCRIPTION OF THE FIGURES

FIG. 1. Identities of deduced amino acid sequences of BBK32 among the isolates of Finnish B. burgdorferi sensu stricto (Bbia), B. garanii (Bg40, Bg46, and Bg50), and B. afzelii (BaA91, Ba1082, Ba570, and Ba600). The identities (%) were calculated from the sequences of the entire proteins including the leader peptides with Multiple sequence alignment, Jotun Hein method, Lasergene software.

FIG. 2. Evaluation of sensitivity of BBK32 in IgG Western blotting (WB) for serodiagnosis of early Lyme borreliosis. Serum samples were collected from culture or PCR-positive patients with erythema migrans at diagnosis (acute) and 1-3 months after antibiotic treatment (convalescent). Immunoreactivity was assessed by densitometry with MacBas 2.5 software. The cut-off for positive WB was defined as the mean value plus 3 SD of 5 healthy blood donors. In IgM and IgG anti-flagellin ELISA (Dako, Denmark), the cut-off value was based on the mean OD plus 3 SD of healthy controls. Ba=B. afzelii; Bg=B. garinii; ND=not performed. +,−=positive or negative WB or ELISA, respectively, by the method indicated.

FIG. 3. IgG ELISA OD values with recombinant BBK32 as an antigen from B. afzelii (panel A), B. garinii (panel B), and B. burgdorferi sensu stricto (Bbia, panel C) with serum samples from patients with neuroborreliosis (NB) or Lyme arthritis (LA). Control samples were from patients with syphilis (SY), systemic lupus erythematosus (SLE), Epstein-Barr virus (EBV) infection, positive rheumatoid factor (RF+), positive for anti-streptolysin (ASO), and samples from healthy blood donors (BD). The cut-off level (mean+3 SD of BD samples) is indicated with a line.

FIG. 4. IgG ELISA OD values with recombinant BBK32 as an antigen from B. afzelii (panel A), B. garinii (panel B), and B. burgdorferi sensu stricto (Bbia, panel C) with serum samples from erythema migrans patients at the acute (EM I) and convalescent (EM II) phases. Control samples were from patients with syphilis (SY), Epstein Barr virus (EBV) infection, positive rheumatoid factor (RF+), and samples from healthy blood donors (BD). The cut-off level (mean+3 SD of BD samples) is indicated with a line.

FIG. 5. ELISA OD/cut-off values of patients with erythema migrans from Finland (FIN), Germany (GER), USA, and Slovenia (SLO). Serum samples were drawn at diagnosis (a) and after antibiotic treatment in convalescence (c). IgG antibodies to rBBK32 from B. afzelii (BBK32-afz), B. garinii (BBK32-gar), B. burgdorferi sensu stricto (BBK32-sensu stricto), and to IR₆ peptide were assessed. Control samples (CO) were from 40 healthy blood donors. The level of positivity for OD/cut-off values (>1) is indicated with a horizontal line.

FIG. 6. IgG antibodies LISA) to the recombinant BBK32 in the CSF of patients with confirmed, probable or possible neuroborreliosis, and of controls, including patients with syphilis, other neurological diseases, and with no proven infection. The level of positivity for OD/cutoff values (>1) is indicated by horizontal lines. The highest OD value of individual CSF sample with the BBK32 variants in ELISAs was used in the analyses.

FIG. 7. IgG antibodies to the recombinant BBK32 in the CSF of patients with duration of neurologic symptoms <3 months (acute) and >3 months (chronic). The level of positivity for the OD/cutoff values (>1) is indicated by a horizontal line. The highest OD value of individual CSF sample with the BBK32 variants in ELISAs was used in the analyses.

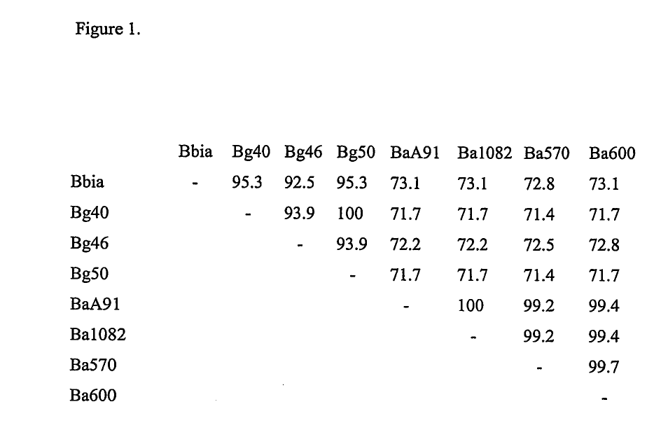

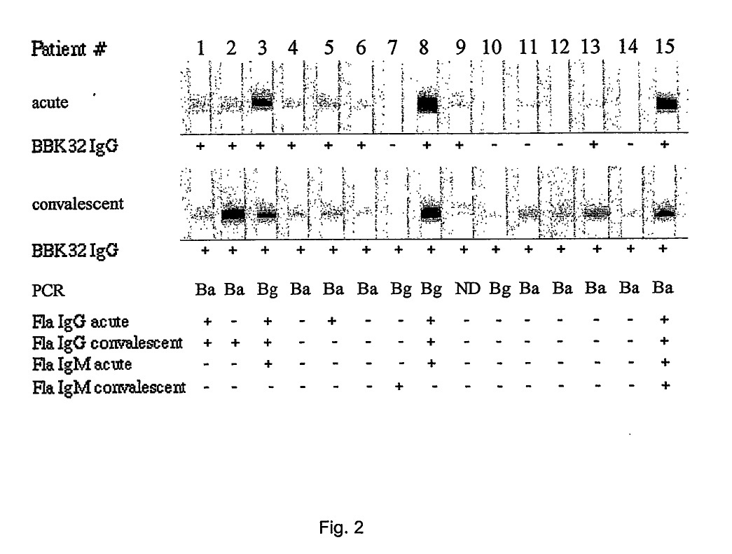

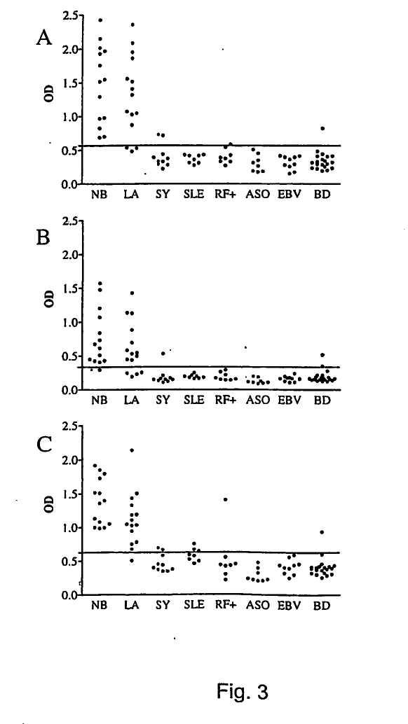

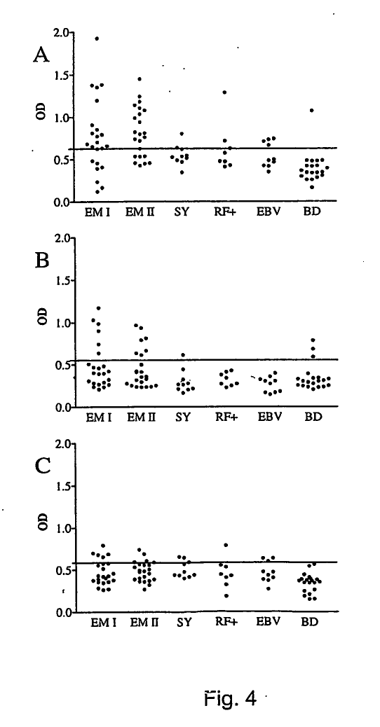

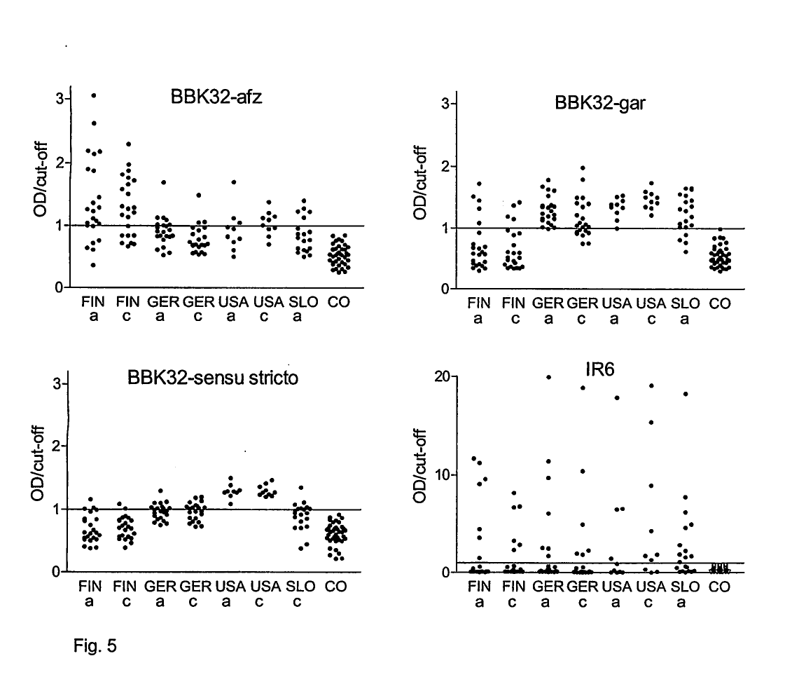

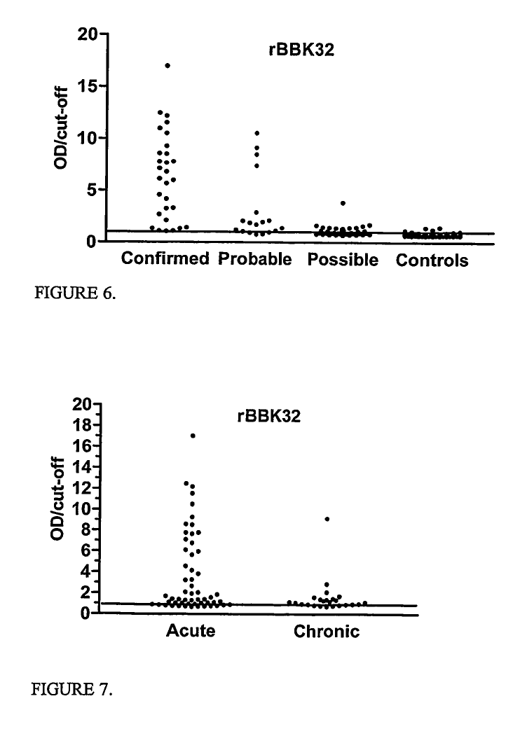

### REFERENCES

1. Steere A C. Lyme disease. N Engl J Med 1989; 321:586-596.
2. Johnsson B J B, Robbins, Bailey R E, et al. Serodiagnosis of Lyme disease: accuracy of a two-step approach using a flagella-based ELISA and immunoblotting. J Infect Dis 1996; 174:346-353.
3. Robertson J, Guy E, Andrews N, et al. A European multicenter study of immunoblotting in serodiagnosis of Lyme borreliosis. J Clin Microbiol 2000; 38:2097-2102.
4. Baranton G, Postic D, Saint Girons I, et al. Delineation of Borrelia burgdorferi sensu stricto, Borrelia garinii sp. Nov., and group VS461 associated with Lyme borreliosis. Int J Syst Bacteriol 1992; 42:378-383.
5. Roessler D, Hauser U, Wilske B. Heterogeneity of BmpA (P39) among European isolates of Borrelia burgdorferi sensu lato and influence of interspecies variability on serodiagnosis. J. Clin. Microbiol. 1997; 35:2752-2758.
6. Mitchell P D, Reed K D, Aspeslet T L, Vandermause M F, Melski J W. Comparison of four immunoserologic assays for detection of antibodies to Borrelia burgdorferi in patients with culture-positive erythema migrans. J Clin Microbiol 1994; 32:1958-1962.
7. Oksi J, Uksila J, Marjamäki M, et al. Antibodies against whole sonicated Borrelia burgdorferi spirochetes, 41-kilodalton flagellin, and P39 protein in patients with PCR- or culture-proven late Lyme borreliosis. J Clin Microbiol 1995; 33:2260-2264.
8. Brown S L, Hansen S L, Langone J J. Role of serology in the diagnosis of Lyme disease. JAMA 1999; 282:62-66.
9. Fikrig E, Barthold S W, Sun W. et al. Borrelia burgdorferi P35 and P37 proteins, expressed in vivo, elicit protective immunity. Immunity 1997; 6:531-539.
10. Zhang J R, Hardman J M, Barbour A G, Norris S J. Antigenic variation in Lyme disease borreliae by promiscuous recombinant VMP-like sequence cassettes. Cell 1997; 89:275-285.
11. Hefty P S, Jolliff S E, Caimano M, et al. Regulation of OspE-related, OspF-related, and E1p lipoproteins of Borrelia burgdorferi strain 297 by mammalian host-specific signals. Infect Immun 2001; 69:3618-3627.
12. Fikrig E, Feng W, Barthold S W, Telford III S R, Flavell R A. Arthropod- and host-specific Borrelia burgdorferi bbk32 expression and the inhibition of spirochete transmission. J Immunol 2000; 164:5344-5351.
13. Akin E, McHugh G L, Flavell R A, Fikrig E, Steere A C. The immunoglobulin (IgG) antibody response to OspA and OspB correlates with severe and prolonged Lyme arthritis and the IgG response to P35 correlates with mild and brief arthritis. Infect Immun 1999; 67:173-181.
14. Magnarelli L A, Ijdo J W, Padula S J, et al. Serologic diagnosis of Lyme borreliosis by using enzyme-linked immunosorbent assays with recombinant antigens. J Clin Microbiol 2000; 38:1735-1739.
15. Rauer S, Spohn N, Rasiah C, et al. Enzyme-linked immunosorbent assay using recombinant OspC and the internal 14-kDa flagellin fragment for serodiagnosis of early Lyme disease. J Clin Microbiol 1998; 36:857-861.
16. Lawrenz M B, Hardham J M, Owens R T, et al. Human antibody responses to VlsE antigenic variation protein of Borrelia burgdorferi. J Clin Microbiol 1999; 37:3997-4004.
17. Mathiesen M J, Christiansen M, Hansen K, et al. Peptide-based OspC enzyme-linked immunosorbent assay for serodiagnosis of Lyme borreliosis. J Clin Microbiol 1998; 36:3474-3479.
18. Liang F T, Steere A C, Marques A R, et al. Sensitive and specific serodiagnosis of Lyme disease by enzyme-linked immunosorbent assay with a peptide based on an immunodominant conserved region of Borrelia burgdorferi VlsE. J Clin Microbiol 1999; 37:3990-3996.
19. Gomes-Solecki M J, Worinser G P, Persing D H, et al. A first-tier rapid assay for the serodiagnosis of Borrelia burgdorferi infection. Arch Intern Med 2001; 161:2015-2020.
20. Fikrig E, Feng W, Aversa J, Schoen R T, Plavell R A. Differential expression of Borrelia burgdorferi genes during erythema migrans and Lyme arthritis. J Inf Dis 1998; 178:1198-1201.
21. Probert W S, Johnson B J. Identification of a 47 kDa fibronectin-binding protein expressed by Borrelia burgdorferi isolate B31. Mol Microbiol 1998; 30:1003-1015.
22. Engstrom S M, Shoop E, Johnson R C. Immunoblot interpretation criteria for serodiagnosis of early Lyme disease. J Clin Microbiol 1995; 33:419-427.
23. Aguero-Rosenfeld M E, Nowakowski J, McKenna D F, Carbonaro C A, Wormser G P. Serodiagnosis in early Lyme disease. J Clin Microbiol 1993; 31:3090-3095.
24. Grodzicki R L, Steere A C. Comparison of immunoblotting and indirect enzyme-linked immunosorbent assay using different antigen preparations for diagnosing early Lyme disease. J Inf Dis 1988; 157:790-797.
25. Aguero-Rosenfeld M E, Nowakowski J, Bittker S, Cooper D, Nadelman R B, Wormser G P. Evolution of the serologic response to Borrelia burgdorferi in treated patients with culture-confirmed erythema migrans. J Clin Microbiol 1996; 34:1-9.
26. Arnez M, Ruzic-Sabljic E, Ahcan J, Radsel-Medvescek A, Pleterski-Rigler D, Strle F. Isolation of Borrelia burgdorferi sensu lato from blood of children with solitary erythema migrans. Pediatr Infect Dis J 2001; 20:251-255.
27. Ornstein K, Berglund J, Nilsson I, Norrby R, Bergström S. Characterization of Lyme borreliosis isolates from patients with erythema migrans and neuroborreliosis in southern Sweden. J Clin Microbiol 2001; 39:1294-1298.
28. Wang G, van Dam A P, Schwartz I, Dankert J. Molecular typing of Borrelia burgdorferi sensu lato: taxonomic, epidemiological, and clinical implications. Clin Microbiol Rev 1999; 12:633-653.
29. Junttila J, Peltomaa M, Soini H, Marjamäki M, Viljanen M K. Prevalence of Borrelia burgdorferi in Ixodes ricinus ticks in urban recreational areas of Helsinki. J Clin Microbiol 1999; 37:1361-1365.
30. Hubalek Z, Halouzka J. Distribution of Borrelia burgdorferi sensu lato genomic groups in Europe, a review. Eur J Epidemiol 1997; 13:951-957.
31. Seppälä IJT, Kroneld R, Schauman K, Forsen K O, Lassenius R Diagnosis of Lyme borreliosis: non-specific serological reactions with Borrelia burgdorferi sonicate antigen caused by IgG2 antibodies. J Med Microbiol 1994; 40:293-302.
32. Wharton M, Chorba T L, Vogt R L, Morse D L, Buehler J W. Case definitions for public health surveillance. Morbid Mortal Weekly Report 1990; 39:19-21.
33. Oksi J, Marttila H, Soini H, Aho H, Uksila J, Viljanen M K. Early dissemination of Borrelia burgdorferi without generalized symptoms in patients with erythema migrans. APMIS 2001; 109: 581-588.
34. Smith R P, Schoen R T, Rahn D W, et al. Clinical characteristics and treatment outcome of early Lyme disease in patients with microbiologically confirmed erythema migrans. Ann Intern Med 2002; 136: 421-428.
35. Huppertz HI, Böhme M, Standaert S M, Karch H, Plotkin S A. Incidence of Lyme borreliosis in the Wurzburg region of Germany. Eur J Clin Microbiol Infect Dis 1999; 18: 697-703.
36. Vaz A, Glickstein L, Field J A, et al. Cellular and humoral immune responses to Borrelia burgdorferi antigens in patients with culture-positive early Lyme disease. Infect Immun 2001; 69: 7437-7444.
37. Strle F, Nelson J A, Ruzic-Sabljic E, et al. European Lyme borreliosis; 231 culture-confirmed cases involving patients with erythema migrans. Clin Inf Dis 1996; 23: 61-65.

1. A method for detecting Borrelia burgdorferi sensu lato infection or the presence of antibodies against Borrelia species in a body fluid from a suspected infected or vaccinated human or other mammal, characterized in that recombinant BBK32 proteins or fragments thereof derived from at least two Borrelia species selected from the group consisting of B. burgdorferi sensu stricto, B. afzelii, and B. garinii are used as antigens in an immunoassay.

2. The method according to claim 1 for the serodiagnosis of early and late Lyme borreliosis.

3. The method according to claim 2 for the serodiagnosis of erythema migrans.

4. The method according to claim 1, characterized in that recombinant BBK32 proteins or their fragments and any other immunogenic protein(s) or their fragments are used as antigens in an immunoassay.

5. The method according to claim 4, characterized in that recombinant BBK32 proteins or their fragments and recombinant decorin binding protein As (DbpAs) or their fragments are used an antigens in an immunoassay.

6. The method according to claim 1, wherein the recombinant BBK32 proteins, optional other immunogenic proteins, or their fragments are derived from B. burgdorferi sensu stricto, B. afzelii, and B. garinii.

7. The method according to claim 6 wherein the recombinant BBK32 proteins, optional other immunogenic proteins, or their fragments are used as antigens either in parallel or combined in an immunoassay.

8. The method according to claim 7 wherein recombinant BBK32 proteins, optional other immunogenic proteins, or their fragments are used together with borrelial peptides.

9. The method according to claim 1, comprising the steps of
a) contacting the body fluid with recombinant BBK32 proteins, optional other immunogenic proteins, or their fragments derived from at least two Borrelia species selected from the group consisting of B. burgdorferi sensu stricto, B. afzelii, and B. garinii, under conditions effective to allow the formation of antigen-antibody complexes; and
b) detecting the complexes formed.

10. The method according to claim 9, characterized in that the body fluid is a serum, plasma, whole blood, cerebrospinal fluid, or synovial fluid sample.

11. A diagnostic kit useful for detecting Borrelia burgdorferi sensu lato infection or the presence of antibodies against Borrelia species in a body fluid from a suspected infected or vaccinated human or other mammal, said kit comprising in a suitable container
a) recombinant BBK32 proteins or their fragments and optionally other immunogenic proteins or their fragments from at least two Borrelia species selected from the group consisting of B. burgdorferi sensu stricto, B. afzelii, and B. garinii, and a detectable label or marker linked to said proteins and/or fragments, or
b) recombinant BBK32 proteins or their fragments and optionally other immunogenic proteins or their fragments from at least two Borrelia species selected from the group consisting of B. burgdorferi sensu stricto, B. afzelii, and B. garinii, and a second antibody linked to any detectable label or marker.

12. The diagnostic kit according to claim 11 for the serodiagnosis of early and late Lyme borreliosis, especially for the serodiagnosis of erythema migrans.

13. The diagnostic kit according to claim 11, wherein the kit comprises recombinant decorin binding protein As (DbpAs).

14. An immunoassay method for diagnosing early and late Lyme borreliosis comprising the steps of
a) contacting a body fluid from a human or other mammal with recombinant BBK32 proteins or their fragments and optionally with other immunogenic proteins or their fragments derived from at least two Borrelia species selected from the group consisting of B. burgdorferi sensu stricto, B. afzelii, and B. garinii, under conditions effective to allow the formation of antigen-antibody complexes; and
b) detecting the complexes found.

15. The immunoassay method according to claim 14 for the serodiagnosis of erythema migrans.

16. The immunoassay method according to claim 14, wherein recombinant BBK32 proteins and recombinant decorin binding protein As (DbpAs) are used as antigens.
